(19) United States
(12) Patent Application Publication
(10) Pub. No.: US 2016/0206637 A1
(43) Pub. Date: Jul. 21, 2016

(54) Effects of Alpha-Glycerophosphocholine Versus Caffeine in Measures of Cognitive, Psychological and Physiological Functions

(71) Applicant: Chemi Nutra, Austin, TX (US)

(72) Inventors: Lorenzo De Ferra, Patrica (IT); Scott L. Hagerman, Austin, TX (US); Martin Purpura, Austin, TX (US); Ralf Jaeger, Austin, TX (US); Chase Hagerman, Austin, TX (US); Maurizio Zenoni, Patrica (IT)

(21) Appl. No.: 14/993,926

(22) Filed: Jan. 12, 2016

Related U.S. Application Data
(60) Provisional application No. 62/102,217, filed on Jan. 12, 2015.

(51) Int. Cl. A61K 31/685; A61K 9/00

(52) U.S. Cl. CPC A61K 31/685; A61K 9/0053; A23L 1/293; A23V 2002/00

(57) ABSTRACT

Alpha-glycerophosphocholine can replace and/or displace caffeine wherever caffeine is used for its physiological benefits in dietary supplements, in beverages, including energy drinks and shots, and in foods and medical foods.

This U.S. Non-Provisional application claim priority to and the benefit from U.S. Provisional Application 62/102,217 filed on Jan. 12, 2015 incorporated herein by reference it its entirety.

### BACKGROUND OF THE INVENTION

α-Glycerophosphocholine (α-GPC) is a nutritional supplement that is converted to phosphorylcholine after ingestion.

Once converted to phosphorylcholine, it has the ability to provide a source of choline for synthesis and release of acetylcholine (ACh), which is the predominant neurotransmitter in the parasympathetic nervous system. ACh is responsible for initiating all skeletal muscle contractions and is involved as a neurotransmitter in various areas of the central nervous system (Brownawell et al, 2011). Due to the effects α-GPC has on ACh synthesis and release, α-GPC may positively affect cognitive function and physiological performance (Hoffman et al., 2010; Shields et al., 2014, Ziegenfuss et al., 2008).

After oral administration, α-GPC is converted to phosphorylcholine, which is a metabolically active form of choline. Phosphorylcholine migrates to the synaptic nerve endings found throughout the entire central nervous system, and in turn increases Ach synthesis and release. Ach is a vastly important neurotransmitter present in both brain and muscle tissue. In the brain ACh plays a key role in basically every cognitive function, while in muscle, it is vitally involved in muscle contraction, as it is the major neuro-transmitter involved in regulating physiological response to exercise.

Starting in the spinal cord, motor neurons branch along their axons and come in contact with muscle fibers at their motor units, where ACh is called upon to deliver the action potential to each muscle fiber at the motor end plate, thus initiating muscle contraction. Motor units are recruited in order of their size (Size Principle), starting with the smallest in size and weakest in degree of tension they can generate. According to this principle, the recruitment sequence begins with Type I motor units (slow twitch, fatigue-resistant, highly oxidative), progresses to Type IIa motor units (fast twitch, fatigue-resistant, oxidative and glycolytic), and finally to Type IIb motor units (fast twitch, fatigable, glycolytic).

It has been demonstrated through scientific study, that engaging in intense exercise can cause a significant reduction in plasma choline levels, thus reducing global stores of ACh, and causing a negative impact on endurance and muscular performance. From a physiological standpoint, this is best explained by the fact that the diminishment of ACh substantially compromises the continual firing of motor units and contraction of “movement specific” muscle groups.

Further, as it can be hypothesized that since all muscle movements—explosive power output, agility, jumping ability—are related to contraction, and contraction is related to available ACh stores, then maximizing ACh should optimize every type of muscular performance.

Caffeine is a mild stimulant that may be found in the leaves, fruits, and/or seeds of many plants such as, for example, Thea species, Camellia species, Theobroma cacao, Coffee arabica, and Cola species. The most common sources of caffeine include coffee (e.g., seeds of Coffee arabica), tea (e.g., leaves of Thea sinensis, Camellia sinensis, etc.), cola soft drinks (e.g., extracts of the nuts of Cola acuminata, Cola nitida, etc.), chocolate (e.g., the seeds of Theobroma cacao), and over-the-counter medications.

Caffeine is recognized as having many physiological and/or pharmacological effects: it may, for example, stimulate the central nervous system, promote analgesia, temporarily increase metabolic function, relax smooth muscle, act as a diuretic, improve running performance, aiding in recovery, providing cognitive support and focus and improving weight lifting abilities, such as increasing power output and strength

Tarnopolsky et al. Medicine and Science in Sports and Exercise, 1989, 21(4): 418-424. Graham, T E. (1998) Effects of Caffeine on Metabolism, Exercise Endurance and Catecholamine Responses and Withdrawl. London. Spriet et al. Caffeine and performance. Int J Sport Nutr. 1995. June:5 Suppl:S84-99. Anderson et al. Improved 2000-meter rowing performance in competitive oarsmen after caffeine ingestion. International Journal of Sport Nutrition and Exercise Metabolism, 2000, 10(4):464-475.

Doherty et al. The effects of caffeine on the maximal accumulated oxygen deficit and short-term running performance. Int J Sport Nutr. 1998; 8:95-104. Armstrong, L. E. Caffeine, body fluid-electrolyte balance, and exercise performance. Int J Sport Nutr Exerc Metab. 2002; June; 12(2):189-206. Hodgson et al. The Metabolic and Performance Effects of Caffeine Compared to Coffee during Endurance Exercise. PloS One, 2013; 8(4): e59561 (published online). Hogervorst et al. Caffeine improves physical and cognitive performance during exhaustive exercise. Medicine and Science in Sports and Exercise, 40 (10), 1841-1851.

The ability of caffeine to stimulate the central nervous system and its ergogenic effects in short term and long endurance are some of the reasons for the popularity of caffeine-containing beverages, dietary supplements and foods. (e.g., caffeinated soft drinks like Red Bull®, Monster Energy®, shots like 5-hour Energy®, and weight loss dietary supplements like Hydroxycut Hardcore®)

There are many reasons that people turn to caffeinated beverages and/or caffeine-containing stimulants products as to promote alertness and physical endurance. Recuperation from certain illnesses may leave one drowsy. Many jobs require a high level of alertness and/or overnight working hours in which drowsiness on the job may be dangerous to the worker and/or others (e.g., police officer, security guard, over-the-road truck driver, etc.). The demands of a school- or job-related deadline may force one to remain alert working or studying into the night. The use of caffeine to increase alertness typically involves ingesting a “dose” of caffeine so that it is absorbed into the bloodstream through the lining of the digestive tract.

A six-ounce cup of coffee can contain from about 40 milligrams (mg) to more than 150 mg of caffeine. A generally accepted “average” caffeine content for a cup of coffee is 100 mg, although many coffee-based drinks typically contain many times that amount. For example, espresso may contain 100 mg of caffeine per fluid ounce. Also, many popular coffee drinks are sold in sizes much larger than the generally accepted “average” six fluid ounce serving.

Certain soft drinks and energy drinks can contain from about 35 mg to about 80 mg of caffeine per serving and, like the coffee-based beverages just described, are often sold in sizes larger than a single serving. Chocolate can contain up to about 15 mg of caffeine per ounce. Certain over-the-counter stimulants contain 100 mg to 200 mg of caffeine.

Despite their popularity, ingestible forms of caffeine such as certain foods, caffeinated beverages, and ingestible over-the-counter medications may not be suitable for all instances in which one might desire to use caffeine to stay alert. For example, caffeinated beverages and over-the-counter medications can require the co-ingestion of fluids, sometimes in large volume. When combined with the diuretic effect of caffeine, this can result in diuresis or an undesirable frequency of urination, particularly if one desires to remain alert in either an environment in which adequate facilities are unavailable or circumstances in which urination would be inconvenient.

Moreover, caffeine has fallen under scrutiny, especially when consumed in dietary supplements, beverages (e.g. energy drinks) and foods by children, adolescents and sensitive adults. Specifically, individuals are concerns about caffeine and its addictive qualities, long-term complete metabolic elimination, and detrimental physical and mental effects including trembling, heart rate increase, insomnia, chronic muscle tension, anxiety, and irritability, and even toxicity.

In addition to these effects, numerous studies have documented caffeine's ergogenic effect on athletic performance, particularly in regard to endurance. Tarnopolsky. Caffeine and endurance. Sports Med. 1994 August; 18(2):109-25. Pasman et al. The effect of different dosages of caffeine on endurance performance time. Int J Sports Med. 1995 May; 16(4):225-30.

Studies have shown that caffeine ingestion prior to exercising extended endurance in moderately strenuous aerobic activity. Goldstein et al. International society of sports nutrition position stand: caffeine and performance. Journal of the International Society of Sports Nutrition 2010, 7:5.

Other studies researching caffeine consumption on elite distance runners and distance swimmers show increased performance times following caffeine consumption. MacIntosh et al. Caffeine Ingestion and Performance of a 1,500-Metre Swim. Canadian Journal of Applied Physiology, 1995, 20(2): 168-177. Stephen et al. Caffeine and Exercise Performance. Sports Medicine January 1993, Volume 15, Issue 1, pp 14-23.

It has now been found that α-GPC, acting as a potent donor of choline, and the subsequent biosynthesis of acetylcholine (ACh), the body's primary neurotransmitter, acts similarly to caffeine. Hoffman J R, et al. The effects of acute and prolonged CRAM supplementation on reaction time and subjective measures of focus and alertness in healthy college students. J Int Soc Sports Nutr. (2010). Shields et al. The effects of a multi-ingredient cognitive formula on alertness, focus, motivation, calmness and psychomotor performance in comparison to caffeine and placebo. Journal of the International Society of Sports Nutrition 2014, 11(Suppl 1):P45.

Therefore α-GPC can replace and/or displace caffeine wherever caffeine is used for its physiological benefits in dietary supplements, in beverages, including energy drinks and shots, and in foods and medical foods.

### SUMMARY OF THE INVENTION

The purpose of this investigation is to determine the acute effects of α-GPC on cognitive, psychological, and physiological functions compared to caffeine and a placebo.

### BRIEF DESCRIPTION OF DRAWINGS

FIG. 1 shows the effects of αGPC-L, αGPC-H and caffeine in comparison to placebo in the Serial Subtraction Test.

FIG. 2 shows the effects of αGPC-L, αGPC-H and caffeine in comparison to placebo on vertical jump peak power;

FIG. 3 shows the results on the VAS for jitteriness.

FIG. 4 shows the flowchart of the procedure described in Experiment 2

FIG. 5 shows the change in isometric Mid-Thigh Pull Peak force after 6 days of supplementation of α-GPC.

FIG. 6 shows the change in Upper-body Isometric Test force after 6 days of supplementation of α-GPC.

### DETAILED DESCRIPTION OF THE INVENTION

Accordingly, the presently claimed invention relates to compositions and methods for the administrations of α-GPC in energy drinks, shots, foods and medical foods replacing and/or displacing caffeine wherever caffeine is used for it physiological benefits.

In one embodiment, the invention provides for oral ingestion of α-GPC to replace caffeine in dietary supplements, beverages and foods.

In another embodiment, the invention also provides for oral ingestion of α-GPC to displace, replace or augment caffeine in dietary supplements, beverages and foods.

The invention provides for oral ingestion of α-GPC to improve explosive power output in relation to physical exercise.

The invention also provides for oral ingestion of α-GPC to improve abilities in combative sports and similar activities. A combative sports describes a contact sport with one-one combat in an individual, for example boxing, kickboxing, amateur wrestling, judo, Brazilian Jujitsu, mixed martial arts, and Muay Thaias, as well as part of a team sport with contact, for example American Football, Rugby, Ice hockey, Soccer.

The invention further provides for oral ingestion of α-GPC to improve cognitive abilities, learning, recall, focus and name and number recognition. Focus describes a person, mentally and physically, having a state or condition permitting clear perception or understanding. Better focus improves the person's center of activity, attraction, or attention, conducting more than one mental or physical task. Another definition describes focus as having or giving the proper sharpness of outline.

The invention also relates to oral ingestion of α-GPC to improve running and sprinting speed, to improve agility and balance and to improve speed up recovery from repeated physical exercise. Agility is the ability to change the body's position efficiently, and requires the integration of isolated movement skills using a combination of balance, coordination, speed, reflexes, strength, and endurance. Agility is the ability to change the direction of the body in an efficient and effective manner and to achieve this requires a combination of balance—the ability to maintain equilibrium when stationary or moving (i.e. not to fall over) through the coordinated actions of our sensory functions (eyes, ears and the proprioceptive organs in our joints); static balance—the ability to retain the center of mass above the base of support in a stationary position; dynamic balance—the ability to maintain balance with body movement; speed—the ability to move all or part of the body quickly; strength—the ability of a muscle or muscle group to overcome a resistance; and lastly, co-ordination—the ability to control the movement of the body in co-operation with the body's sensory functions (e.g., in catching a ball [ball, hand and eye co-ordination]). In sports, agility is often defined in terms of an individual sport, due to it being an integration of many components each used differently (specific to all of sorts of different sports). Sheppard and Young (2006) defined agility as a “rapid whole body movement with change of velocity or direction in response to a stimulus”. Agility is also an important attribute in many role playing games, both computer games and as Dungeons and Dragons. Agility may affect the character's ability to evade an attack or navigate uneven terrain.

The invention further relates to oral ingestion of α-GPC for increasing workload and enhance training to improve physical performance. Physical performance relates to the ability to complete certain physical tasks with higher intensity, faster, or with a higher power output. Improvement, maintenance or reduced loss of physical performance may be a beneficial physiological effect for individuals performing physical exercise for different reasons (e.g. athletes preparing for a competition or during a competition, and individuals engaged in physical work or recreational activities), but also for individuals performing common (non-exercise-related) physical tasks. Information on the characteristics (e.g. type, duration and intensity) of the exercise or physical activity for which the claim is made may be important for the definition of the claimed effect (e.g. physical performance during short-term, high intensity exercise vs. longer term, endurance performance; single exercise bout vs. repeated bouts; weight bearing vs. non-weight bearing activities) and of the target population for the claim (e.g. athletes and elderly subjects). Outcome measures of physical performance which may be appropriate for the assessment of the claimed effect in humans in the context of a particular type of exercise or physical activity should be indicated (e.g. time spent to run a certain distance, distance cycled during a time-trial, throwing distance in javelin or shot put, jumping height, walking speed and number of chair-stands in a certain time). Some of the outcomes proposed (e.g. changes in maximum oxygen consumption (VO2max), muscle glycogen stores and substrate oxidation) are not direct measures of performance but could be used in support of a mechanism by which the food/constituent could exert the claimed effect on physical performance.

The invention further relates to oral ingestion of α-GPC to improve reaction time in relation to daily activities and in physical exercise.

The invention is directed to compositions and methods for the administrations of α-GPC in energy drinks, shots, foods and medical foods for replacing and/or displacing caffeine wherever caffeine is used for it physiological benefits.

Formulations containing an oral dosage of between about 20 mg to about-600 mg of α-GPC can be used to affect mental and physical performance. The formulations of α-GPC can be included in dietary supplements, beverages and foods.

An oral dosage of between about 20 mg to about 600 mg of α-GPC can be used to replace and/or displace from about 50 mg to about 300 mg of caffeine.

This experimental protocol is given in detail in order to more clearly explain how to make and use the invention and do not limit the scope of the appended claims.

Approximately 20 recreationally active participants (10 males and 10 females) between the ages of 18 and 29 years of age were recruited to participate in this study.

Participants were not allowed to take part in this research if they meet any of the following criteria: 1) have current or past history of anabolic steroid use; 2) have any metabolic disorders including known electrolyte abnormalities; heart disease, arrhythmias, diabetes, thyroid disease or hypogonadism; a history of hypertension, hepatorenal, musculoskeletal, autoimmune, or neurologic disease; if they are taking thyroid, hyperlipidemic, hypoglycemic, anti-hypertensive, or androgenic medications; 3) have ingested any ergogenic levels of creatine, HMB, thermogenics, ribose, pro-hormones (i.e., DHEA, androstendione, etc.) or other purported anabolic or ergogenic nutritional supplements for a 1-month time period prior to beginning the study and to not take any additional nutritional supplement (except a vitamin/mineral supplement) or contraindicated prescription medication during the protocol. All participants meeting entrance criteria signed informed consent statements in compliance with the Human Participants Guidelines of Angelo State University.

Independent and Dependent Variables

The independent variables used throughout this study were the nutritional supplementation and the number of testing/evaluation times during the study. Dependent variables included cognitive function, mood, reaction time, hand-eye coordination, power, speed, and agility.

Entry and Familiarization Session

Participants expressing interest in participating in this study were interviewed to determine whether they appeared to qualify to participate in this study. Participants believed to meet eligibility criteria were invited to attend an entry/familiarization session. During this session, participants signed Informed Consent Statements and complete medical histories. Participants meeting entry criteria were familiarized to the study protocol via a verbal and written explanation outlining the study design. This included describing the supplementation protocol, familiarizing the participants to the tests to be performed, and practicing all testing procedures.

Testing Protocol

Participants arrived at the Human Performance Laboratory at Angelo State University having fasted for at least 8 hours prior to the testing protocol. Participants were also asked to refrain from consuming any food or beverages that contain caffeine or other stimulants for at least 24 hours prior to each testing session. Following supplementation, participants performed all non-fatiguing tests first. These tests included resting heart rate using a Polar heart rate monitor (Polar Electro, Inc., Lake Success, N.Y.) and blood pressure (aneroid sphygmomanometer), the serial subtraction test to measure cognitive function, visual analog scales (motivation, calmness, focus, fatigue, vigor, and jitteriness), the online reaction time test (www.humanbenchmark.com/tests/reactiontime/), and the hand-eye coordination test. For the serial subtraction test, participants were asked to repeatedly subtract the number 7 from a random 4-digit number for a period of 2 minutes. Participants were scored by average time per correct calculation and percentage of correct calculations. The visual analog test were administered by asking the participants to indicate how they feel on a linear scale from 0-10 by making a vertical line on a 10 cm long horizontal line. The distance from 0 was measured using a metric ruler.

The online reaction time test required the participants to click a mouse button when the computer screen changes color from red to green. The average reaction time over 5 attempts was recorded. For the hand-eye coordination test, participants were asked to bounce a tennis ball off of a wall from a distance of 2 meters. The participant were required to throw the ball with their right hand, catch it in their left hand, throw the ball with their left hand, and catch the ball with their right hand. This was repeated for a total of 30 seconds.

Participants were scored by the total number of successful catches. Following the non-fatiguing tests, the participants performed a standardized warm-up in which they walked on a treadmill for 5 minutes at 3 miles per hour. Following the warm-up, participants performed the fatiguing tests which included vertical jump, broad jump, 40 yard dash, and the pro-agility shuttle. During the vertical jump test, participants performed three attempts and had a Tendo Unit (Tendo Sport Machines, Trencin, Slovak Republic) attached to their waist to measure maximum velocity and power output in addition to measuring their vertical jump height using a Vertec Jump Measurement System (Jump USA, Sunnyvale, Calif.). Participants then performed three attempts for the standing broad jump. Maximum broad jump distance was recorded. Lastly, participants performed one 40 yard dash and one pro-agility shuttle (5-10-5 drill) on an indoor, wooden gymnasium floor. Times was measured by hand and recorded.

Supplementation Protocol

Upon arrival to the Human Performance Laboratory at Angelo State University on each of the 4 testing sessions, participants were provided with a sample of the supplement. Participants were assigned to consume a placebo, 200 mg of caffeine, 200 mg of α-GPC, and 400 mg of α-GPC in a randomized, double-blind, placebo controlled, cross-over manner. After consuming the supplement, participants waited 30 minutes before completing the testing protocol. All participants were advised that they should not have had anything to eat or drink, except for water, in the previous 8 hours. Additionally, participants were asked to refrain from consuming any food or beverages that contain caffeine or other stimulants for at least 24 hours prior to completing the testing sessions.

| FAM Session | Supplement 1 | Supplement 2 | Supplement 3 | Supplement 4 |
|---|---|---|---|---|
| Familiarization | Consume Randomly | Consume Randomly | Consume Randomly | Consume Randomly |
| session, | Assigned | Assigned | Assigned | Assigned |
| complete | supplement | supplement | supplement | supplement |
| paperwork. | | | | |
| Review medical | | | | |
| history | | | | |
| Complete all | Perform non- | Perform non- | Perform non- | Perform non- |
| familiarization | fatiguing tests (SST, | fatiguing tests (SST, | fatiguing tests (SST, | fatiguing tests (SST, |
| practice tests | VAS, reaction time, | VAS, reaction time, | VAS, reaction time, | VAS, reaction time, |
| for all | hand-eye | hand-eye | hand-eye | hand-eye |
| dependent | coordination) | coordination) | coordination) | coordination) |
| variables | Perform | Perform | Perform | Perform |
| | standardized warmup | standardized warmup | standardized warmup | standardized warmup |
| | Perform all other | Perform all other | Perform all other | Perform all other |
| | tests (vertical jump, | tests (vertical jump, | tests (vertical jump, | tests (vertical jump, |
| | broad jump, 40 yard | broad jump, 40 yard | broad jump, 40 yard | broad jump, 40 yard |
| | dash, pro-agility | dash, pro-agility | dash, pro-agility | dash, pro-agility |
| | shuttle) | shuttle) | shuttle) | shuttle) |

### Example 1

### Methods

Twenty participants (10 males, 10 females; 22.0±3.4 years of age; height 171.9±7.4 cm; weight 56.8±8.6 kg) consumed 200 mg of αGPC (αGPC-L, Alpha-Size®, Chemi Nutra, Austin, Tex. USA), 400 mg of Alpha-GPC (αGPC-H), 200 mg of caffeine (CA), and a placebo (PL) in a randomized, double-blind, placebo-controlled, crossover design. Participants performed the following measurements 30 minutes after supplementation: visual analog scales (VAS) for six different moods, a serial subtraction test (SST), and tests for reaction time, hand-eye coordination, power, speed and agility.

### Results

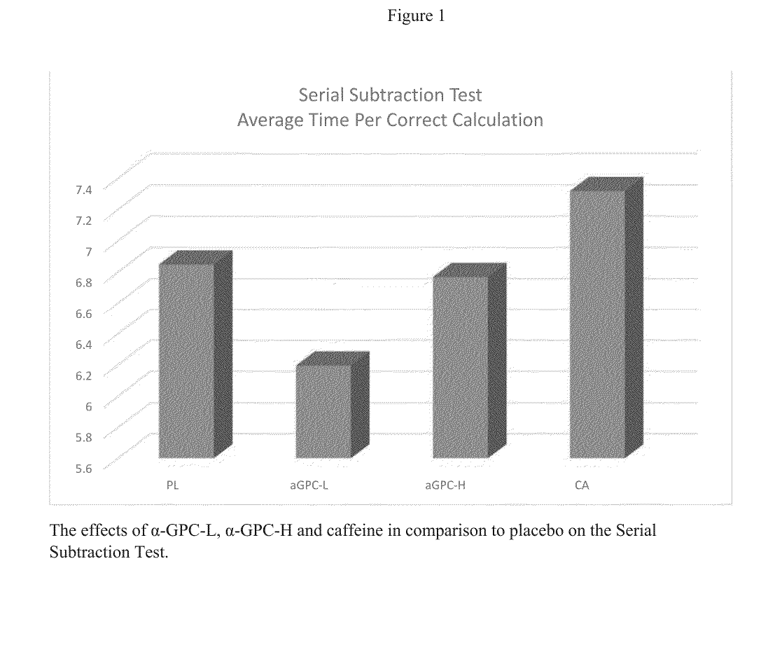

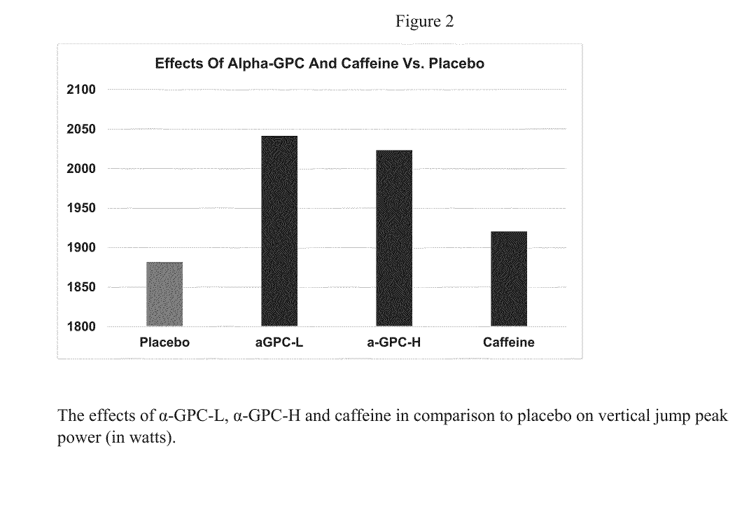

SST scores were 18.1% and 10.5% faster in the αGPC-L (6.19±2.21 s) group compared to CA (7.32±5.67 s) and PL (6.85±2.52 s), respectively (FIG. 1). Vertical Jump Peak Power was 8.5% higher in the αGPC-L (2,041.3±547.2 W), 7.5% higher in the αGPC-H (2,023.1±942.8 W) and 2.0% higher in the CA group (1,920.4±689.6 W) in comparison to PL (1,881.9±689.6 W) (FIG. 2).

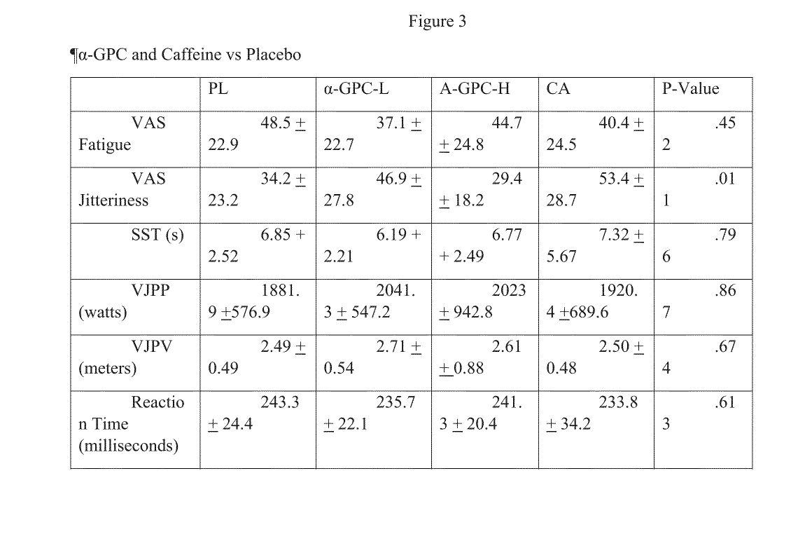

The group consuming CA had significantly higher scores on the VAS for jitteriness compared to αGPC-H, but not αGPC-L or PL (FIG. 3).

### Conclusion

α-GPC seemed to be beneficial for certain physical and mental performance tasks and is at least as effective as CA, without displaying the negative side effects of CA supplementation

### Example 2

Ergogenic aides are widely used by fitness enthusiasts and athletes to increase performance. α-GPC has demonstrated some initial promise in changing explosive performance. The purpose of the present investigation was to determine if 6 days of supplementation with α-GPC would augment isometric force production compared to a placebo.

### Methods

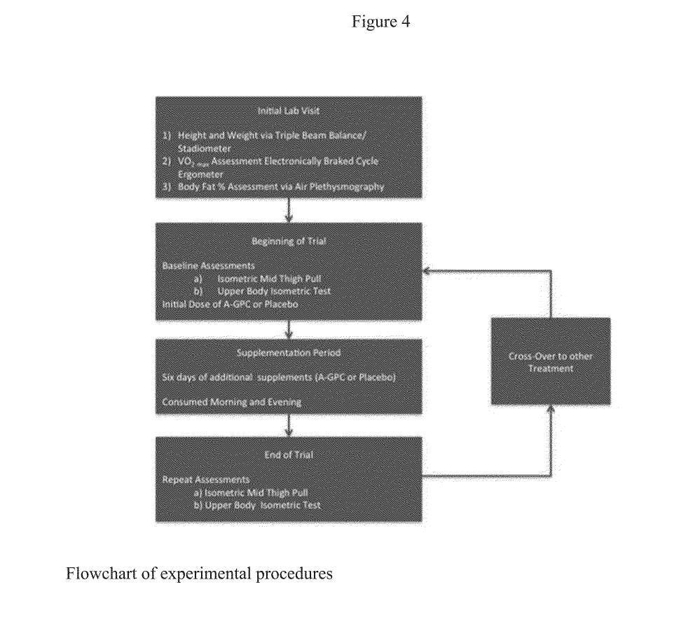

The Institutional Review Board at the University of Louisiana at Lafayette reviewed the present investigation for ethics. The study was a double-blind, placebo-controlled crossover with a 1-week washout period that included 13 healthy, college-aged males (Means±SD; Age: 21.9±2.2 years, Height: 180.3±7.7 cm; Weight: 87.6±15.6 kg, VO₂ max: 40.08±7.23 ml O₂*kg⁻¹; body fat: 17.5±4.6%). Subjects reported to the lab and give informed consent, which included consent to publish, prior to baseline assessments, which included height and weight, an assessment of maximum aerobic capacity via a COSMED CPET system (COSMED, Rome ITL) with integrated electronically braked cycle ergometer as outlined in previous studies incorporated herein by reference (5) and body fat percentage via air displacement plethysmography (Bod Pod Gold Standard System, COSMED Rome, ITL). The following week trial one (random order: either placebo or 600 mg of αGPC) began. For the trials baseline performance testing was done and they were given an initial dose (placebo or α-GPC) while in the lab, 1 h later the performance testing (isometric mid-thigh pull, upper body isometric test) was repeated. The subjects were then given 6 days of additional pre-packaged supplement to take (morning and evening). The subjects reported back on day 6 of this period to repeat performance testing after the final dose of supplement. After a 1-week washout period, the subjects repeated the trial with the other treatment (see FIG. 4).

### Treatments

The treatments consisted of 600 mg daily of α-GPC (Alphasize®, ChemiNutra, Austin Tex.) or a placebo. Both treatments were administered in the same capsules (gel caps) and were the same color (white). The α-GPC capsules were supplied with a certificate of analysis from a third party lab confirming the amount of active ingredient. The placebo capsule consisted of microcrystalline cellulose and magnesium stearate (Nature's Supplements, Carlsbad, Calif. USA). Both the participant and researcher were unaware of the identity of either treatment until the end of the study. The participants were instructed to take doses in the morning and evening that would deliver a total of 600 mg of α-GPC per day and were given the pills in a non-distinct plastic bottle marked only with a code. The participants returned the bottles at the end of the study.

The participants reported 100% compliance with taking the required doses.

### Isometric Mid-Thigh Pull (IMTP)

The isometric mid-thigh pull test (IMTP) is a well validated strength measure (Beckman, G, et al. “Relationship of isometric mid-thigh pull variables to weightlifting performance.” J. Sports Med. Phys Fitness 2010; 35:573-81). Testing was conducted in a customized power rack (Rogue Fitness, Columbus, USA) that is secured to a concrete laboratory floor surrounding a AMT1 Force Plate (Advanced Materials Technologies Inc., Watertown USA). The power rack allows for small incremental adjustments in height for a steel bar that is secured via two large tubular steel members. The participant was instructed to stand with the feet shoulder width apart above the force plate. The height of the bar was adjusted so that the participant was in a position where the torso was upright (assessed via a contractors box level), the knees achieved between 120-130° of flexion (measured via a goniometer) and the arms were straight while holding the bar. The participants were told to “drive straight up” and to pull as hard as they could against the chain until the force began to noticeably decline. The peak force was assessed at a sampling rate of 2000 Hz using an AMT1 Force Plate. Subjects were familiarized with the IMTP during the initial lab visit. Measurements were taken in triplicate with a five-minute rest.

### Upper Body Isometric Test (UBIST)

The participants were positioned on three elevated platforms with the chest directly suspended over a load cell anchored into the concrete floor of the lab (iLoad Pro, Loadstar Sensors, Fremont Calif.). The load cell had a capacity of greater than 5000 r and a listed accuracy of 0.25% for the full scale of measurement. The participants were placed in a push-up style position, with the hands at 150% of biacromial width, and the elbows at 90° of extension (measured via a goniometer). A thick, non-elastic strap was run over one shoulder and under the opposite shoulder and connected with metal rings to a chain that was tethered to the load cell. The participants were instructed to keep their backs flat, and push with their hands maximally until told to stop by the researcher. Prior to data capture the load cell was tared to ensure the weight of the load cell and apparatus were accounted for. The researcher started data collection and verbally instructed the participant to “push as hard as possible”. The participants were verbally encouraged during data collection, which was terminated when the force production declined by 50 N from the peak value registered. The load cell was set to capture data at maximum rate (150 Hz) and the data was exported and analyzed in JMP 11.0 (SAS Institute Inc, Ca1y NC). Peak force values were isolated from the data and used for subsequent analysis. The test was performed three times with 5 min rest between assessments. The validity and reliability of this test have been reported in the literature.

### Statistical Analysis

Reliability was assessed for the isometric tests via Intra Class Correlation Coefficients (ICC). Repeated measures Anovas were used to examine acute (baseline and 1 h post) and chronic (baseline and day 6) changes in performance between treatments. Order of administration (Placebo first, A-GPC first) was entered into the model as a covariate. G* Power software was used to determine effect size (Cohen's d), all other analyses were performed using a modern statistical software package (JMP, version 11.0 SAS Institute Inc., Caty, N.C.). Magnitude based inferences were calculated to assist with interpretation of results. The use of magnitude based inference is an attempt to expand the interpretation of findings to include harmful, trivial and beneficial as interpretations, rather than just significant, nonsignificant. This interpretations in not without controversy as such the authors have chosen to include it along-side a more traditional statistical approach.

### Results

### Reliability of Isometric Tests

The isometric tests demonstrated reliability when the triplicate measurements were examined via ICC (range: 0.969-0.984). Measurements were not different at any time points (p>0.05). Therefore in subsequent analysis the peak value from the set of three measures was used.

### Treatment Effects-Acute

Repeated measures Anova did not reveal any main effects (F=0.003, p=0.9584) nor interaction effects of treatment time (F=0.114, p=0.738) for IMTP performance 1 h after the initial close of A-GPC or Placebo. Similar results were revealed with UBIST performance was analyzed.

### Treatment Effects-Chronic

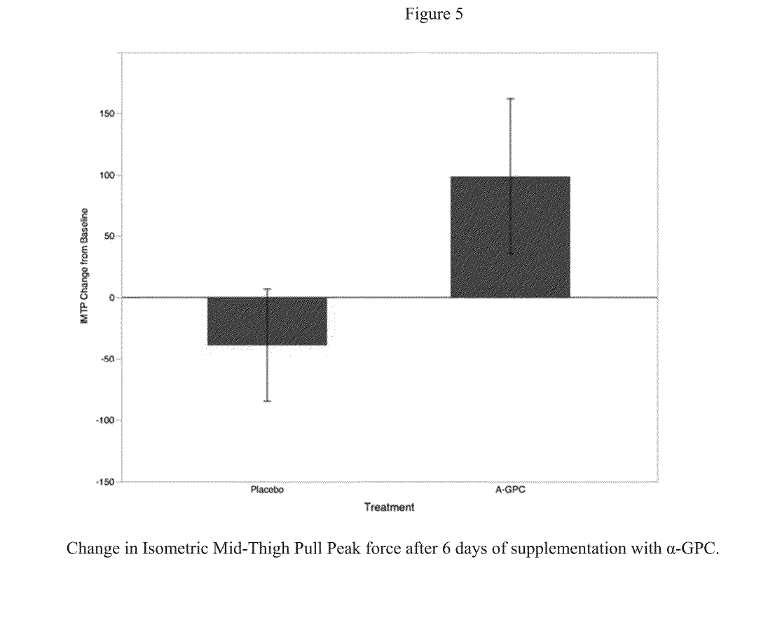

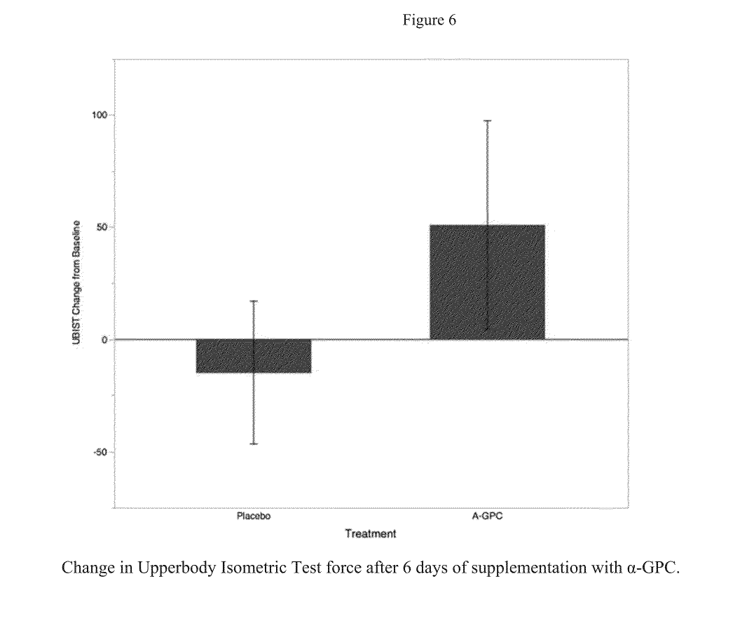

Repeated measures Anova revealed a significant interaction effect for treatment (A-GPC vs Placebo) by time (baseline, day 6) for lMTP peak performance (F=3.12, p=0.04; change from baseline A-GPC: 98.8.±236.9 N vs Placebo: −39.0±170.9 N, ES=0.961). See FIG. 5. For the upper body test the α-GPC treatment trended towards greater change from baseline force production (A-GPC: 50.9±167.2 N Placebo: −14.9±114.9 N) but the interaction effect of treatment by time failed to obtain statistical significance (F=1.36, p=0.127). However, this data (see FIG. 6) demonstrated a large effect size (ES=0.714). This suggests that the variability of the subject's upper body strength limited the statistical power, however, it if likely that a real effect exists in this data. Magnitude based inferences suggest that the α-GPC was 68.3% likely beneficial for increasing upper body isometric force and 86.5% likely beneficial for increasing lower body isometric force production.

### Discussion

The results of this study support the use of A-GPC to enhance strength, particularly in the lower body. The literature does not contain controlled experimental data regarding the effects of A-GPC on aspects of human performance directly related to strength, and thus this study represents a first step in the evaluation of this product for such use. The literature does contain some evidence that choline itself is important to consider in regard to endurance performance. While further studies will be needed to confirm the results reported from this experiment, the data represent a promising start and suggest alternative uses for A-GPC.

The potential mechanism by which A-GPC could confer enhanced strength and power performance involves increased bio-available choline, which may results in augmented acetylcholine synthesis in neurons. A-GPC has been shown to augment acetylcholine levels in CNS neurons. Several studies have shown that when administered either intramuscularly or orally A-GPC can increase plasma choline levels. A-GPC has also been shown to increase growth hormone secretion though the action of acetylcholine stimulated catecholamine release. This increase in cholinergic tone and associated increased growth hormone release was also reported in old and young subjects after administration of growth hormone releasing hormone in conjunction with A-GPC.

While the present study presents positive preliminary findings for A-GPC augmenting strength, it is not without limitation. The study will need to be replicated with alternative measures of human performance, likely those that have the capacity to measure power not just peak force. Additionally, different does of A-GPC need to be explored to determine any potential dose-response, or lower limit for meaningful effect. We suggest that in vitro studies may also be warranted to demonstrate that A-GPC has the potential to augment neurotransmitter levels in motor neurons.

### CONCLUSIONS

The results of the study suggest that A-GPC is effective at increasing lower body force production after 6 days of supplementation. A similar trend was noted in upper body isometric strength, however; this failed to attain statistical significance. Given that in many sports it is understood that a very small change in performance, often times less than 2 percent, can significantly affect outcomes it is important to note that the 6 days of A-GPC resulted in greater than a 3 percent increase in lower body isometric strength. Sport performance coaches can consider adding A-GPC to the diet of speed and power athletes to enhance muscle performance.

### REFERENCES

1. Brownawell, A. M., Carmines, E. L., Montesano, F. Safety assessment of α-GPC as a food ingredient. Food and Chemical Toxicology 49(2011). 1303-1315.
2. Hoffman, J. R., Ratamess, N. A., Gonzalez, A., Beller, N. A., Hoffman, M. W., Olxon, M., Purpura, M., Jäger, R. The effects of acute and prolonged CRAM supplementation on reaction time and subjective measures of focus and alertness in healthy college students. Journal of the International Society of Sports Nutrition 2010, 7(39).
3. Shields, K. A., Silva, J. E., Rauch, J. T., Lowery, R. P., Ormes, J. A., Sharp, M. H., McCleary, S. A., Georges, J., Joy, J. M., Purpura, M., Jager, R., Wilson, J. M. The effects of a multi-ingredient cognitive formula on alertness, focus, motivation, calmness, and psychomotor performance in comparison to caffeine and a placebo. Journal of the International Society of Sports Nutrition 2014, 11(Suppl 1):P45
4. Ziegenfuss, T., Landis, J., Hofheins, J., Acute supplementation with alpha-glycerylphosphorylcholine augments growth hormone response to, and peak force production during, resistance exercise. Journal of the International Society of Sports Nutrition 2008. 5(Suppl 1): P15.
5. Ryan E J et al., “Low-dose caffeine administered in chewing gum does not enchance cycling to exhaustion. J. Strength Con. Res. 2012, 26(4):1154-61.
Ryan E J, Kim C H, Muller M D, Bellar D M, Barkley J E, Bliss M V, Jankowski-Wilkinson A, Russell M, Otterstetter R, Macander D, Glickman E L, Kamimori G H. Low-dose caffeine administered in chewing gum does not enhance cycling to exhaustion. J Strength Cond Res. 2012; 26(4):1154-1161.
6. Beckham, G., Mizuguchi, S., Carter, C., Sato, K., Ramsey, M., Lamont, H., Horsby, G., Haff, G., Stone, M., 2010. Relationship of isometric mid-thigh pull variables to weighlifting performance. J Sports Med Phys Fitness. 35, 573-581.
7. Faul F, Erdfelder E, Lang A-G & Buchner A. G*Power 3: A flexible statistical power analysis program for the social, behavioral, and biomedical sciences. Behavior Research Methods. 2007; 39:175-191.
8. Batterham A M, Hopkins W G. Making meaningful inferences about magnitudes. Int J Sports Physiol Perform. 2006; 1(1):50-57.
9. Conlay L A, Wurtman R J, Blusztajn J K, Coviella I L G, Maher T J, Evoniuk G E. Decreased plasma choline concentrations in marathon runners. N Engl J Med. 1986; 315:982.
10. Penry J T, Manore M M. Choline: an important micronutrient for maximal endurance-exercise performance? Int J Sport Nutr Exerc Metab. 2008; 18:191-203
11. Gatti G, Barzaghi N, Acuto G, Abbiati G, Fossati T, Perucca E. A comparative study of free plasma choline levels following intramuscular administration of L-alpha-glycerylphosphorylcholine and citicoline in normal volunteers. Int J Clin Pharmacol Ther Toxicol. 1992; 30(9):331-335.
12. Kawamura T, Okubo T, Sato K, Fujita S, Goto K, Hamaoka T, Iemitsu M. Glycerophosphocholine enhances growth hormone secretion and fat oxidation in young adults. Nutrition. 2012; 28:1122-1126.
13. Ceda G P, Ceresini G, Denti L, Marzani G, Piovani G, Banchini A, Tarditi E, Valenti G. Horm Metab Res. 1992; 24(3): 119-121.

1. A method of replacing physiological effects of caffeine contained in dietary supplements, beverages and foods in a subject, said method comprising:
orally administering to said subject dietary supplements, beverages and foods in which an effective amount of α-glycerophosphocholine (α-GPC) substitutes said caffeine; and
obtaining said physiological effects in said subject.

2. The method of claim 1, wherein said effective amount comprises between about 20 mg to about 600 mg of α-GPC.

3. The method of claim 2, wherein said effective amount displaces from about 50 mg to about 300 mg of caffeine.

4. The method of claim 1, wherein said effective amount of α-GPC displaces or augments said physiological effects of caffeine in said subject.

5. The method of claim 1, wherein said α-GPC provides for cognitive and/or physical benefits similar to caffeine without caffeine's detrimental physical and mental effects.

6. The method of claim 5, wherein said detrimental physical and mental effects comprise anxiety, depression, jittery and crash.

7. A method of improving explosive power output in relation to physical exercise in a subject, said method comprising:
orally administering to said subject dietary supplements, beverages and foods in which an effective amount of α-GPC substitutes caffeine; and
improving said explosive power output in relation to physical exercise in said subject.

8. The method of claim 7, wherein said effective amount comprises between about 20 mg to about 600 mg of α-GPC.

9. The method of claim 7, wherein said effective amount substitutes from about 50 mg to about 300 mg of said caffeine.

10-12. (canceled)

13. A method of improving cognitive abilities selected from the group consisting of learning ability, recall, focus ability and name and number recognition in a subject, said method comprising:
orally administering to said subject dietary supplements, beverages and foods in which an effective amount of α-α-GPC substitutes caffeine; and
improving said cognitive abilities in said subject.

14. The method of claim 13, wherein said effective amount comprises between about 20 mg to about 600 mg of α-GPC.

15. The method of claim 13, wherein said effective amount substitutes from about 50 mg to about 300 mg of caffeine.

16-36. (canceled)
